(19) United States
(12) Patent Application Publication
(10) Pub. No.: US 2026/0135587 A1
(43) Pub. Date: May 14, 2026

(54) Zone Monitor Badge Proximity and Communication System

(71) Applicant: Ecolab USA Inc., Saint Paul, MN (US)

(72) Inventor: David L. Snodgrass, Stuart, FL (US)

(21) Appl. No.: 19/385,817

(22) Filed: Nov. 11, 2025

Related U.S. Application Data
(60) Provisional application No. 63/718,872, filed on Nov. 11, 2024.
(60) Provisional application No. 63/817,834, filed on Jun. 4, 2025.

(51) Int. Cl. H04B 1/7163; G16H 40/67

(52) U.S. Cl. CPC H04B 1/71632; G16H 40/67

(57) ABSTRACT

The present subject matter relates to, among other things, ultra-wideband (UWB) radio hand hygiene compliance monitoring systems and methods that more reliably and robustly communicate in high electromagnetic noise and interference environments, such as clinics, hospitals, and nursing homes. In various applications, the present system employs ranges or angle-of-arrival (AoA) and time-of-flight (ToF) calculations to determine multiple zones about a patient and to provide more granular zones about an area of interest for use in activities, such as hand hygiene compliance monitoring. The present subject matter provides additional customizable zone topologies for monitoring than conventional beacon and badge zone monitoring.

### RELATED APPLICATIONS

The present application claims the benefit of U.S. Provisional Application Ser. No. 63/718,872, filed Nov. 11, 2024, “Zone Monitor Badge Proximity and Communication System and U.S. Provisional Application Ser. No. 63/817,834, filed Jun. 4, 2025, “Zone Monitor Badge Proximity and Communication System.”

The present application incorporates by reference the entire disclosures of: U.S. application Ser. No. 17/654,324 filed Mar. 10, 2022, now U.S. Pat. No. 11,711,745 B2, “Adaptive Route, Bi-Directional Network Communication”; U.S. Provisional Application Ser. No. 63/718,872, filed Nov. 11, 2024, “Zone Monitor Badge Proximity and Communication System; and U.S. Provisional Application Ser. No. 63/817,834, filed Jun. 4, 2025, “Zone Monitor Badge Proximity and Communication System.”

### BACKGROUND

Hand hygiene compliance monitoring (HHCM) in healthcare settings has been a critical focus for improving patient safety and reducing healthcare-associated infections. Early HHCM systems employed near-field radio communications to create proximity boundaries around patient beds and hand hygiene dispensers.

These systems typically used low-speed communications, such as 125 kHz, to detect when a healthcare worker's (HCW) badge entered a near-field boundary, triggering communication with the associated device (bed or dispenser beacon).

Over the past decade, the medical environment, particularly in hospitals, has seen a significant increase in the number of devices and systems utilizing 125 kHz near-field communications.

This proliferation, coupled with the growing presence of patient room electronic equipment, has created a challenging radio frequency environment that interferes with the 125 kHz near-field communications used in HHCM systems.

Existing HHCM systems also employ UHF (917 MHz) communications between radios to determine proximity, but these lack the precision needed for accurate relative proximity measurements.

Moreover, 917 MHz communications, while unlicensed for low-power use, may suffer from similar electromagnetic interference issues as near-field radio systems and potentially introduce interference to medical equipment in proximity to patients.

The increasing electromagnetic noise and interference in healthcare environments have highlighted the limitations of current HHCM technologies. These limitations include, but are not limited to:
1. Susceptibility to interference from other devices using similar frequency ranges.
2. Potential for introducing interference to sensitive medical equipment.
3. Lack of precision in proximity detection and measurement.
4. Challenges in creating accurately defined and shaped monitoring zones.

As the healthcare industry continues to adopt more wireless technologies and electronic equipment, there is a growing need for improved radio communications for hand hygiene systems. Specifically, there is a demand for systems that are more robust and reliable in environments with increasing electromagnetic interference, while also providing enhanced accuracy in proximity detection and zone definition.

There is a need in the art for new technologies that provide more robust communications and improve the effectiveness of hand hygiene compliance monitoring in modern healthcare settings.

### SUMMARY

The present application relates to, among other things, ultra-wideband (UWB) radio hand hygiene compliance monitoring systems and methods that more reliably and robustly communicate in high electromagnetic noise and interference environments, such as clinics, hospitals, and nursing homes. UWB radio is an ideal choice for a hospital environment at least in part due to its very low transmit power and disparate frequency operation. UWB radio communications are less likely to interfere with other technologies such as Wi-Fi and Bluetooth. UWB radio communications also have a low susceptibility to in-band interference from other narrowband technologies such as Wi-Fi and Bluetooth.

In various embodiments, the present system employs angle-of-arrival (AoA) and time-of-flight (ToF) calculations to determine multiple zones about a patient and to provide more granular zones about an area of interest for use in activities, such as hand hygiene compliance monitoring. The present subject matter provides additional customizable zone topologies for monitoring than conventional beacon and badge zone monitoring. The present subject matter allows for sampling of radio signals from a badge or other portable radio device, and processes those sampled signals to produce information related to AoA and/or ToA. In various embodiments, the present system employs a directional antenna of a zone monitor and a UWB radio to determine multiple granular zones about an area of interest for use in activities, such as hand hygiene compliance monitoring. The present subject matter allows for sampling of UWB radio signals from a badge or other portable radio device, and processes those sampled radio signals to produce zone information. The present approach provides lower power consumption and thus longer battery life than techniques requiring multiple measurements.

This Summary is an overview of some of the teachings of the present application and not intended to be an exclusive or exhaustive treatment of the present subject matter. Further details about the present subject matter are found in the detailed description and appended claims. The scope of the present invention is defined by the appended claims and their legal equivalents.

### BRIEF DESCRIPTION OF THE DRAWINGS

In the drawings, which are not necessarily drawn to scale, like numerals may describe similar components in different views. The drawings illustrate generally, by way of example, but not by way of limitation, various embodiments discussed in the present patent application.

FIG. 1 shows a plan view of a patient room layout with a Patient Zone Monitor (PZM), according to one embodiment of the present subject matter.

FIG. 2 is a graph illustrating the emitted signal power across different frequency ranges for various wireless technologies, including UWB (ultra-wideband) radio, synonymous with UWB's relatively low power and relatively wide frequency range.

FIG. 3 depicts a UWB Radio Pulse, showing the normalized voltage over time and illustrating the extremely short pulse time of less than Ins used in UWB communications.

FIG. 4 is a diagram illustrating the time-of-flight (ToF) measurement process between two UWB devices, demonstrating how distance is calculated using signal transmission and reception timing.

FIG. 5 shows a plan view of a patient room layout with the PZM, illustrating the patient zone boundary and maximum range of the PZM, according to various embodiments of the present subject matter.

FIG. 6 shows a more complex patient zone boundary with multiple boundary ranges associated with different arrival angles, demonstrating the system's capability to create a “shaped” patient zone, according to various embodiments of the present subject matter.

FIG. 7 depicts a patient room layout with multiple hand hygiene dispensers, showing the dispenser zone boundaries and maximum ranges for each dispenser.

FIG. 8 illustrates an alternative proximity detection method using RF range to set the zone boundary, showing how the PZM antenna can be designed to shape the RF field and provide an elliptical zone boundary.

FIGS. 9-11 demonstrate portions of a wireless network in which the present subject matter may be employed, according to one embodiment of the present subject matter.

FIG. 12 is a block diagram illustrating components of a machine, according to some example embodiments, able to read instructions from a machine-readable medium (e.g., a machine-readable storage medium) and perform any one or more of the methodologies discussed herein.

### DETAILED DESCRIPTION

The following detailed description of the present subject matter refers to subject matter in the accompanying drawings which show, by way of illustration, specific aspects and embodiments in which the present subject matter may be practiced. These embodiments are described in sufficient detail to enable those skilled in the art to practice the present subject matter. References to “an”, “one”, or “various” embodiments in this disclosure are not necessarily to the same embodiment, and such references contemplate more than one embodiment. The following detailed description is demonstrative and not to be taken in a limiting sense. The scope of the present subject matter is defined by the appended claims, along with the full scope of legal equivalents to which such claims are entitled.

The present subject matter relates to, among other things, ultra-wideband (UWB) radio hand hygiene compliance monitoring systems and methods that more reliably and robustly communicate in high electromagnetic noise and interference environments, such as clinics, hospitals, and nursing homes.

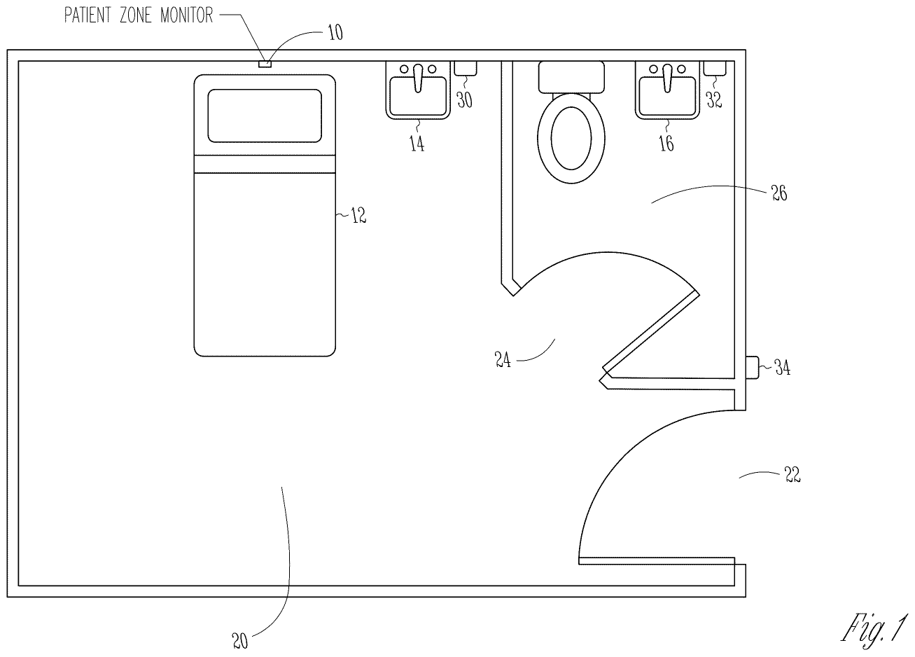

FIG. 1 shows a plan view of a patient room layout equipped with various components for monitoring hand hygiene compliance. The patient room 20 is equipped with a Patient Zone Monitor (PZM) 10 positioned near the patient bed 12 to track entry and exit of persons into a designated patient zone area (not shown in FIG. 1) about the patient bed 12. The room is outfitted with multiple hand hygiene stations 30, 32, and 34, and includes two sinks 14, 16, strategically placed for convenient access. A doorway or entrance 22 provides access to the room 20. The bathroom area 26 is entered through doorway 24 and includes a toilet and sink 16. This comprehensive layout emphasizes the integration of hand hygiene monitoring capabilities with standard patient room features, demonstrating a focus on infection control and compliance with hand hygiene protocols in healthcare settings.

Ideally, a health care worker (HCW) disinfects hands before entering the patient zone area 24. That can be done by using hand hygiene dispenser 34 for disinfecting the hands of the HCW. It can also be done by using the hand hygiene dispenser 30 if it is outside of the patient zone area. The room is also fitted with a hand hygiene dispenser 32 in the bathroom area 26. Hence, designated patient zone area is important to determine if a hand hygiene event is recorded by the system by a HCW to be in compliance with hand hygiene protocols.

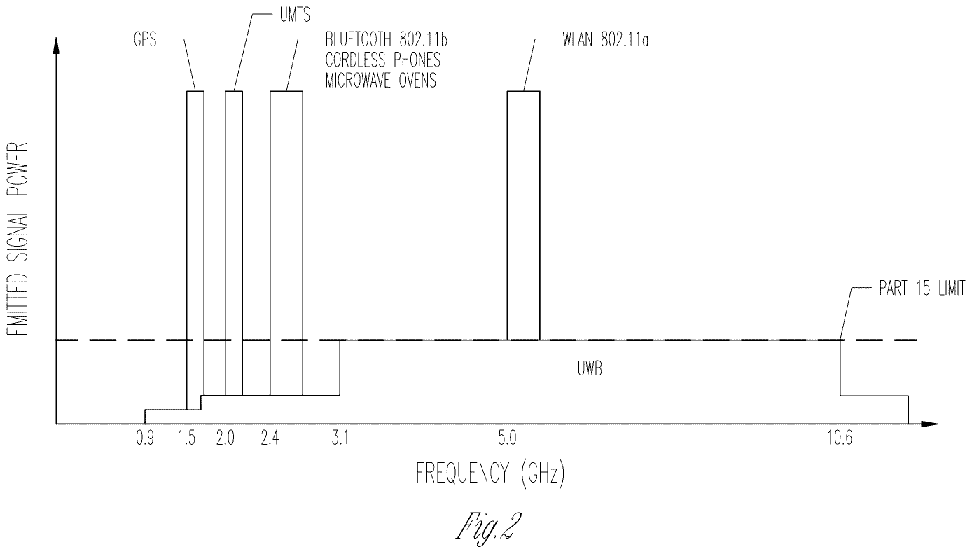

FIG. 2 illustrates a graph comparing the emitted signal power of various wireless technologies across different frequency ranges. The x-axis represents frequency in GHz, ranging from 0.9 to 10.6 GHz, while the y-axis shows the emitted signal power. The graph depicts several technologies, including GPS, UMTS, Bluetooth, 802.11b, cordless phones, microwave ovens, WLAN 802.11a, and UWB (Ultra-Wide Band). A horizontal line labeled “PART 15 LIMIT” is shown, representing a regulatory power limit. The UWB technology is represented by a wide, low-power band spanning from approximately 3.1 to 10.6 GHz, demonstrating its unique characteristic of operating across a broad frequency range at relatively low power levels compared to other technologies.

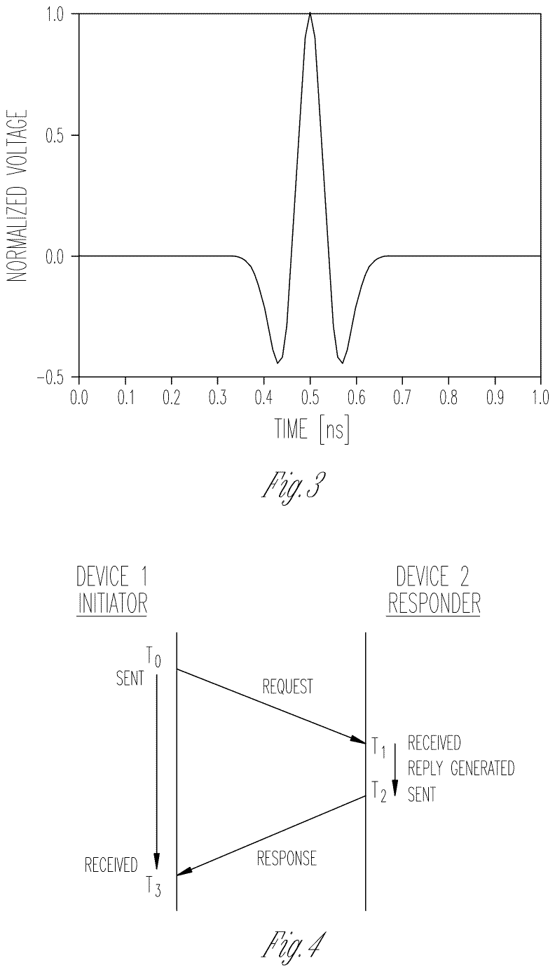

FIG. 3 displays a graph of a normalized voltage pulse over time, characteristic of UWB technology. The x-axis represents time in nanoseconds, ranging from 0 to 1.0 ns, while the y-axis shows normalized voltage from −0.5 to 1.0. The graph depicts a sharp, narrow pulse with a rapid rise to a peak of 1.0, followed by a quick descent to approximately −0.5, and then a return to the baseline. This pulse shape is typical of UWB signals, which use very short duration pulses to transmit information, allowing for high data rates and precise timing measurements.

These figures collectively illustrate the unique properties of UWB technology, highlighting its wide frequency range, low power consumption, and short pulse duration, which are key features for applications in hand hygiene compliance monitoring systems as described in the patent application.

Hospitals, clinics, and assistive care facilities are known to have complex radio frequency environments due to the number of wireless devices in proximity to a patient for monitoring and care of the patient. A number of different wireless standards may be employed. UWB radio is an ideal choice for a hospital environment at least in part due to its very low transmit power and disparate frequency operation. UWB radio communications are less likely to interfere with other technologies such as Wi-Fi and Bluetooth. UWB radio communications also have a low susceptibility to in-band interference from other narrowband technologies such as Wi-Fi and Bluetooth.

UWB radio communications are a wireless communication mode that was originally called “pulse radio.” Around 2002 the US Federal Communication Commission (FCC) released UWB regulations allowing unlicensed use of UWB communications in the allocated spectrum; however, the allowable power limit was set extremely low (−41.3 dBm) to avoid interference with other technologies, such as Wi-Fi, Bluetooth, etc. The low spectral density of UWB signals makes them less susceptible to in-band interference from other narrowband signals and very secure as they are difficult to detect due to low power density. UWB is useful for wirelessly connecting devices over short distances and can outperform Bluetooth because it has superior speed, uses less power, is more secure, and provides superior location discovery and device ranging for hand hygiene monitoring applications.

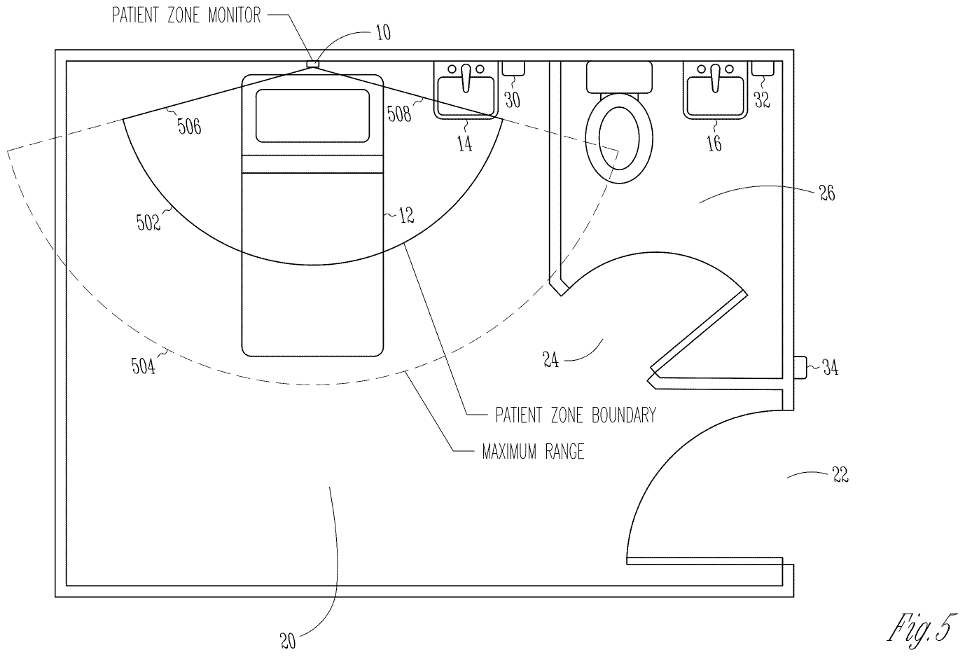

Radios like Bluetooth, Wi-Fi, and others use a modulated stream of sine waves to communicate. UWB radio has the unique ability to use time-of-flight (ToF) measurement to accurately determine distance between radios. UWB radios use a series of very short discrete pulses to communicate. FIG. 4 illustrates the ToF measurement process used by Ultra-Wide Band (UWB) technology to accurately determine the distance between two UWB radio devices.

The diagram shows two devices: Device 1 (Initiator) and Device 2 (Responder), and depicts the communication sequence between them. The process begins with Device 1 sending a request signal at time T₀. This signal travels to Device 2, which receives it at time T₁. Device 2 then generates and sends a reply at time T₂. Finally, Device 1 receives the response at time T₃.

This sequence allows for precise timing measurements between the transmission and reception of signals. By calculating the round-trip time and accounting for processing delays at Device 2, the system can determine the time it takes for the signal to travel between the two devices. Since UWB uses very short, discrete pulses for communication (as opposed to the modulated sine waves used by technologies like Bluetooth and Wi-Fi), it can achieve highly accurate timing measurements.

The ability to precisely measure these time intervals enables UWB technology to calculate the distance between devices with a high degree of accuracy. This feature is particularly valuable in the context of hand hygiene compliance monitoring, as it allows for precise localization of personnel and equipment within a healthcare setting.

In various embodiments, the present system employs angle-of-arrival (AoA) and time-of-flight (ToF) calculations to determine multiple zones about a patient and to provide more granular zones about an area of interest for use in activities, such as hand hygiene compliance monitoring. The present subject matter provides additional customizable zone topologies for monitoring than conventional beacon and badge zone monitoring.

Due to the extremely short pulse time (in some embodiments, the pulse time is less than 1 nanoseconds), the round-trip time a pulse travels from a first radio (Device 1) to a second radio (Device 2) and back again can be measured when the reply time of Device 2 is known (T₂−T1, as demonstrated in FIG. 4), as set forth in Equation 1:

$$ToF = \frac{( {T}_{3} - {T}_{0} ) - ( {T}_{2} - {T}_{1} )}{2} \qquad Eq . 1$$

Once the total ToF (seconds) is known, this value can be multiplied by the speed of light (c=299,792,458 m/s) to determine the distance (meters) between Device 1 and Device 2, as set forth in Equation 2:

$$Distance = ToF \cdot c \qquad Eq . 2$$

Thus, the distance estimate obtained from UWB communications can be used to determine an approximate range of communications between a plurality of UWB radios, and to establish relative position of a tag (for example, on a person) entering or exiting a predetermined zone (for example, the vicinity of a patient bed). Those of skill in the art will appreciate that UWB communications increase the reliability of communications in an environment with electromagnetic interference and additionally provides a mechanism for range estimation using ToF. This allows creation of a system that can logically determine when a person having a UWB radio (such as used in a tag) is in proximity to a predetermined object or scene (e.g., patient bed or patient room).

Existing hand hygiene compliance monitoring systems may also use UHF (917 MHz) communications between radios to determine when a first radio is in proximity to a second radio, but lack the precision needed to determine relative proximity measurement of the radios. 917 MHz communications are similarly unlicensed for use at low power but may suffer from the same electromagnetic interference issues of near field radio systems and may also introduce interference to medical equipment in proximity to a patient.

The present subject matter can employ UWB communications in combination with the near field and UHF radio systems and it can provide modes where UWB replaces one or both of the near field and UHF radio communications, in various embodiments.

In various embodiments, UWB radios in the healthcare hand hygiene compliance monitoring (HHCM) system will completely replace the current system's 125 kHz near-field radios, which are confined to the use of proximity detection. In various embodiments, UWB radios will replace the need for 917 MHz UHF communications between the badge and beacons as the UWB radio can be used for both proximity detection and data communication. For example, in current systems, a badge may support both a 125 kHz near-field receiver and a 917 MHz transceiver. In various embodiments of the present subject matter both radios will be replaced by a single UWB radio.

Also, current bed beacon and dispenser beacons support a 125 KHz near-field transmitter and a 915 MHz/917 MHz UHF transceiver. Using the present subject matter, the 125 KHz near-field transmitter will be replaced by a UWB transceiver. The UHF radio currently provides two functions. The 917 MHz band is used for short range badge communication and the 915 MHz FHSS band is used for long range network communication. With the addition of the UWB radio, the UHF radio can be retained and dedicated to network communication if desired. In such embodiments, badge communication may occur primarily via the UWB radio.

In one exemplary embodiment of the present system, the system will monitor the proximity of a healthcare worker (HCW) in and around established patient zones and hand hygiene dispenser zones. A patient zone is typically the area around a patient bed in a patient room; however, other zones may be used without departing from the scope of the present subject matter. The World Health Organization defines the patient zone as the area within one meter of the center of a patient bed. A hand hygiene dispenser zone is about a minimum of one arm's length (about 24 inches) from the base of a hand hygiene soap/sanitizer dispenser. Other zones are possible without departing from the scope of the present subject matter.

Each monitored HCW will wear a badge. The badge will wirelessly interact (via at least UWB) with patient zone monitor and dispenser zone monitor devices. In various embodiments, the badge will use ToF measurement to determine if it is within a zone. A zone boundary will be established for each zone monitor either by default or during installation. As an example, a patient zone monitor's zone boundary may be set to 50 inches and a dispenser zone monitor's zone boundary may be set to 28 inches. Persons of skill in the art will appreciate that other dimensions, spaces, and configurations may be used without departing from the scope of the present subject matter.

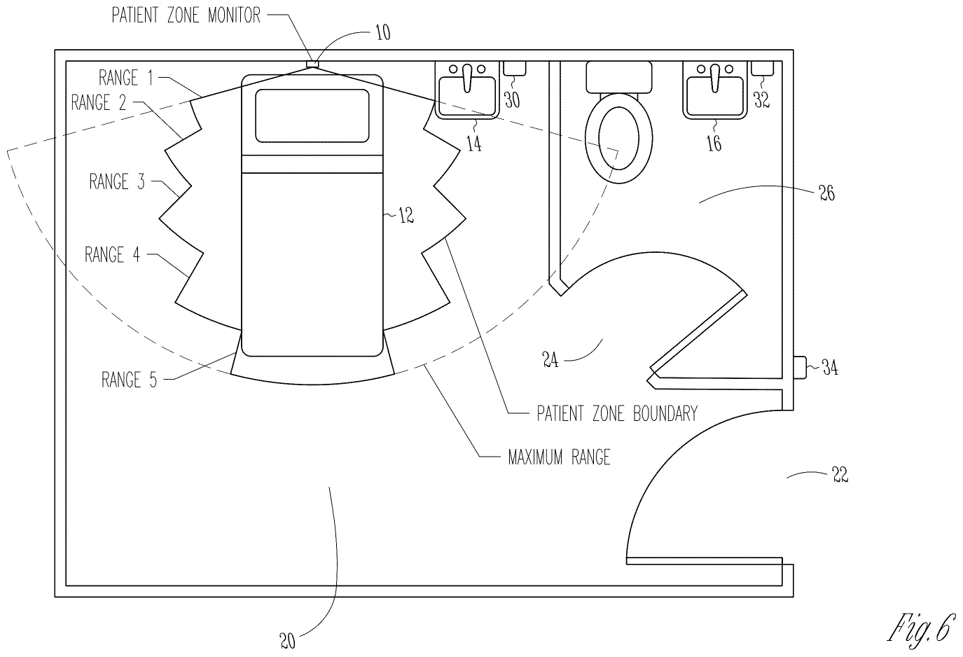

In various embodiments of the system set forth in FIG. 5, the patient zone monitor (PZM) will be mounted on the wall at the head of the patient bed and simultaneously mounted above the patient bed so that a clear line-of-sight (no obstacles) path is available to a HCW's badge. In various embodiments, the PZM includes a directional UWB antenna so transmitted and received signals are directed over the patient bed. In various embodiments, the UWB antenna may be omnidirectional, but exhibit directionality due to materials it is mounted upon, such as a concrete or steel surface that acts like an absorber and/or a reflector of electromagnetic energy so as to make the antenna emission and/or reception patterns directional. This will prevent the PZM from transmitting and receiving signals through the mounting wall and possibly interfering with a PZM and patient bed located on the other side. Both the patient zone boundary 502 and maximum range 504 can be adjusted and/or adjustable to provide adequate isolation. However, the range is substantially equidistant from the PZM at any angle, such as angles 506 and 508, due to the nature of the antennas and the omnidirectional aspect of basic PZM systems.

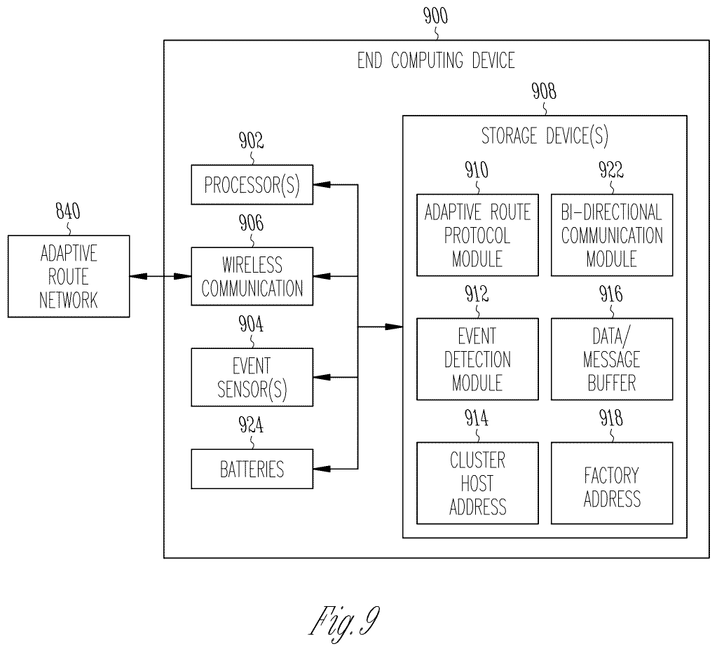

In various embodiments of the system set forth in FIG. 5, the PZM includes a directional UWB antenna so transmitted and received signals are directed over the patient bed. Tailored zones are possible using a directional antenna, such as zone 802 as shown in FIG. 8. FIG. 8 illustrates a proximity detection method using RF range to set the zone boundary. The PZM antenna can be designed to shape the RF field and provide an elliptical zone boundary, such as zones 802 and 804. When the HCW badge is within signal range of the PZM, it is in the patient zone. The PZM antenna can be designed to shape the RF field to provide a variety of shapes, including, but not limited to, an elliptical shape 802, 804. In various embodiments a shaped RF field is preferable over a circular radius.

In various embodiments, the PZM may include a low noise amplifier (LNA) with adjustable gain to provide different zones, such as zones 802 and 804 of FIG. 8. In various applications a single directional antenna may be employed that provides a directionality over the area of interest. In various embodiments, an antenna array may be employed for directionality. In various embodiments a ground plane is used that enhances the directionality of the antenna and reduces transmission into the wall that the PZM 10 is mounted to. In various embodiments, the PZM uses a 3 inch by 3 inch ground plane to serve as a reflector of the radio frequency signals emanating from the antenna of the PZM 10. Different antennas, ground planes, gain adjustments, and transmission methods may be employed without departing from the scope of the present subject matter.

In embodiments using an LNA, the LNA setting may be adjusted and then saved for a particular environment, such as a hospital or patient room. In various embodiments, the LNA may be adjusted to different settings to control sensitivity of the system within an environment, such as a hospital or patent room.

In one approach to the present system, if a HCW's badge is close enough to receive the zone monitor's transmitted signal, then the system deems the badge within the zone, such as zone 802 or 804. Various badges may be employed, such as ones with a light that illuminates when the badge is within a radio frequency zone of a PZM 10. Such a tool allows for the set up of the PZM 10 within an environment.

Patient zone boundary data will be included in the message exchange between the PZM and the badge. It will typically originate from the PZM. Therefore, each PZM can be given a unique patient zone boundary value or range. There will be a default range in each PZM but that may be adjusted during installation or changed remotely via wireless network.

In one embodiment of the present subject matter, the UWB radios are programmed to support angle-of-arrival (AoA) when implementing at least two antennae separated by a known distance on a receiver that supports AoA. As a signal arrives it will be received by the first antenna. Slightly later, the signal will be received by the second antenna. By measuring the time between signal reception by the first and second antenna, the angular location of the signal's origin, relative to the receiver, can be determined. In various embodiments employing the range approach the need to calibrate ToF for each HCW badge is not necessary and reduces the time and complexity of the HCW badge's firmware calculating ToF distance.

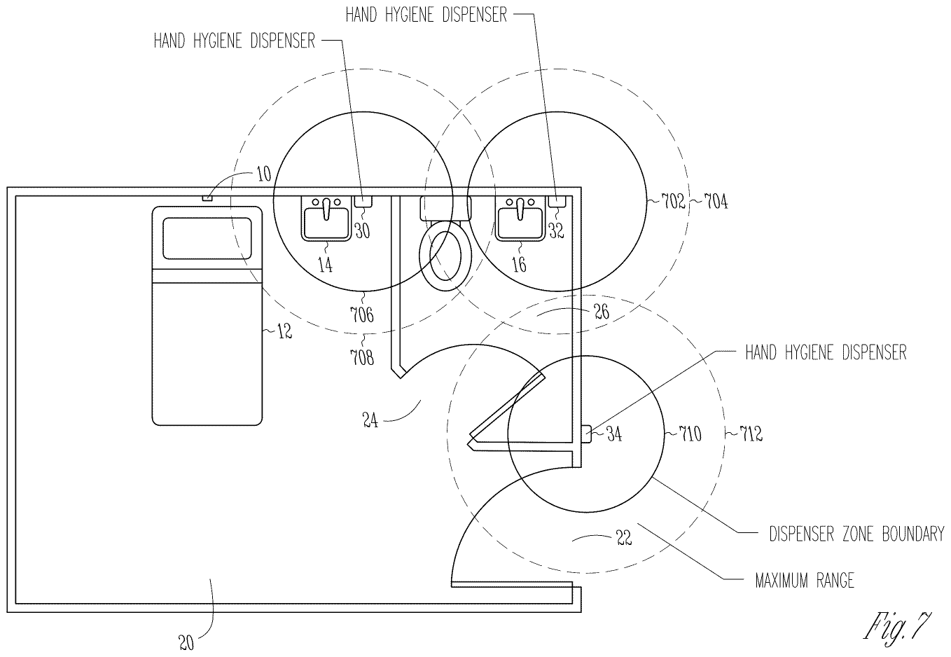

A set of patient zone boundary ranges can be associated with a predetermined span of arrival angles to create a “shaped” patient zone. FIG. 6 shows a more complex patient zone boundary with multiple boundary ranges associated with different arrival angles, demonstrating the system's capability to create a “shaped” patient zone, according to various embodiments of the present subject matter. The example of FIG. 6 demonstrates an example of five boundary ranges that were selected with each assigned to a 15° span of signal arrival angles (Range 1=15° to 30°, Range 2=30° to 45°, Range 3=45° to 60°, Range 4=60° to 75°, and Range 5=75° to) 90°.

Because the patient zone will be symmetrical around the patient bed, i.e., mirrored along the length of the patient bed, arrival angle and boundary range data from only one side needs to be sent to the badge. When an arrival angle greater than 90° occurs, the arrival angle can be normalized by the following calculation set forth in Equation 3:

$$Normalized AoA = 180 ° - AoA \qquad Eq . 3$$

When the HCW badge interacts with the PZM, the PZM's arrival angle and boundary range data will be passed to the badge. The badge will then determine if it is within the patient zone, based on AoA and ToF data, and react accordingly.

The dispenser zone monitor (DZM) will be mounted within manually activated and touch-free activated hand hygiene dispensers. Both the dispenser zone boundary and maximum range can be adjusted. The system can provide improved measurements through the use of a third antenna in a triangular configuration with the two antennae. This allows the system to differentiate signals in front of the PZM as opposed to signals from behind the PZM.

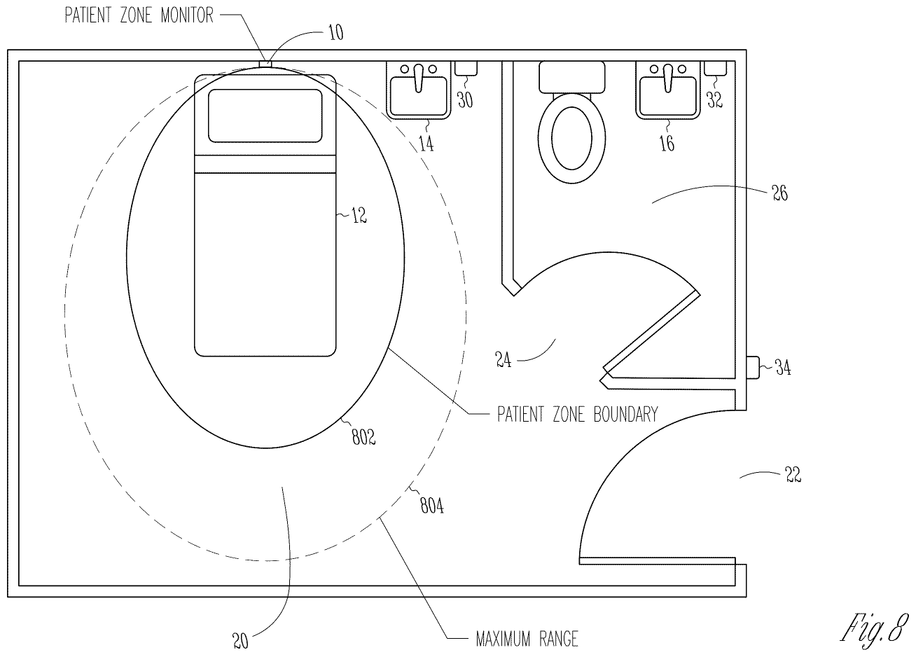

Dispenser zone boundary data will be included in the message exchange between the DZM and the badge. In various embodiments, message exchange may originate from the DZM. FIG. 7 depicts a patient room layout with multiple hand hygiene dispensers, showing the dispenser zone boundaries and maximum ranges for each dispenser. Therefore, each DZM can be given a unique dispenser zone boundary value or range, such as is demonstrated in FIG. 7 by dispenser zone boundaries 702, 706, and 710 (and maximum ranges 704, 708, and 712, respectively). There will be a default range in each DZM but that may be adjusted during installation or changed remotely via wireless network.

The DZM will only be active for a predetermined time (1 second) after the host dispenser has been activated. During this time, the HCW badge will interact with the DZM, the DZM's boundary range data will be passed to the badge. The badge will then determine if it is within the dispenser zone, based on ToF data, and react accordingly.

ToF proximity detection has the limitation that the zone boundary cannot be shaped without the added cost and complexity of AoA. As shown in FIG. 5, the zone boundary will always be a fixed distance from the PZM.

FIG. 8 illustrates an alternative proximity detection method using RF range to set the zone boundary as a variation of the proximity detection method. FIG. 8 shows how the PZM antenna can be designed to shape the RF field and provide an elliptical zone boundary. When the HCW badge is within signal range of the PZM, it is in the patient zone. Additionally, the PZM antenna can be designed to shape the RF field to provide an elliptical shape 802, 804 instead of a fixed circular radius. This method negates the need for ToF calibration for each HCW badge during manufacturing and eliminates the added time and complexity of the HCW badge's firmware calculating ToF distance.

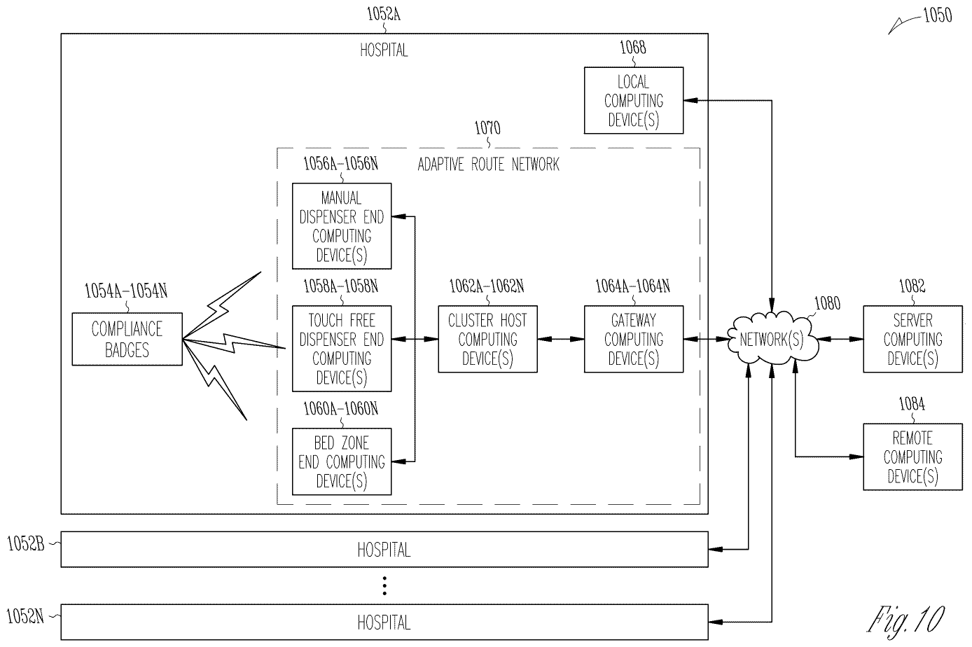

FIGS. 9-11 demonstrate portions of a wireless network in which the present subject matter may be employed, according to one embodiment of the present subject matter. Portions of this system are set forth in U.S. application Ser. No. 17/654,324 filed Mar. 10, 2022, now U.S. Pat. No. 11,711,745 B2, “Adaptive Route, Bi-Directional Network Communication.”

FIG. 9 is a block diagram of an example end computing device 900 in accordance with the adaptive route network and protocol of the present disclosure, connected to network 840. FIG. 9 illustrates only one example of end computing device 900, and many other examples of end computing device 900 may be used in other instances. End computing device 900 may include a subset of the components included in FIG. 9 or may include additional components not shown in FIG. 9.

End computing device 900 includes one or more processor(s) 902, a wireless communication unit 906, one or more event sensor(s) 904, one or more storage device(s) 908, and one or more battery 924. Storage device(s) 908 includes an adaptive route protocol module 910, a bi-directional communication module 922, an event detection module 912, a cluster host address 914, a factory address 918, and data storage/message buffer 916.

One or more wireless communication units 906 of end computing device 900 are configured to permit bi-directional wireless communication with one or more cluster host computing devices. Examples of communication units 906 include any device or technology capable of sending and receiving wireless communications. Such devices may include optical transceivers, radio frequency (RF) transceivers, infrared (IR) transceivers, satellite communication, cellular communication, etc.

One or more processors 902 may implement functionality and/or execute readable instructions associated with end computing device 900. Examples of processors 902 include application processors, microcontrollers, and any other hardware configured to function as a processor, a processing unit, controller, or a processing device.

For example, processors 902 may execute adaptive route protocol module 910 to execute a cluster host discovery process. Processors 902 may further execute bi-directional communication protocol module 922 to transmit downstream message from the end computing device 908 to a gateway and/or to receive upstream messages transmitted from a gateway or server to end device 908. Processors 902 may further execute event detection module 912 to detect events and perform any corresponding analysis or communication regarding such detected events, depending upon the requirements of the application in which the end devices are being implemented.

Adaptive route protocol module 910, event detection module 912, and bi-directional communication module 922, as well as other functional modules not shown in FIG. 9, may be operable by processors 902 to perform various actions, operations, or functions of end computing device 900. For example, processors 902 of end computing device 900 may retrieve and execute instructions stored by storage components 908 that cause processors 902 to perform or execute the operations stored in modules 910, 912 and/or 922. The instructions, when executed by processors 902, may cause end computing device 900 to generate and/or store information within storage components 908, such as data storage/message buffer 916.

In some examples, storage device(s) 908 may include a temporary memory, meaning that a primary purpose of such as portion of storage device(s) 908 is not long-term storage. Storage device(s) 908 on end computing device 900 may be configured for short-term storage of information as volatile memory and therefore not retain stored contents if powered off. Examples of volatile memories include random access memories (RAM), dynamic random-access memories (DRAM), static random-access memories (SRAM), and other forms of volatile memories known in the art.

Storage device(s) 908, in some examples, may also include one or more computer-readable storage media. Storage device(s) 908 in some examples include one or more non-transitory computer-readable storage mediums. Storage device(s) 908 may be configured to store larger amounts of information than typically stored by volatile memory. Storage device(s) 908 may further be configured for long-term storage of information as non-volatile memory space and retain information after power on/off cycles. Examples of non-volatile memories include magnetic hard discs, optical discs, floppy discs, flash memories, or forms of electrically programmable memories (EPROM) or electrically erasable and programmable (EEPROM) memories. Storage device(s) 908 may store program instructions and/or information (e.g., data) associated modules 910, 912 and/or 922. Storage device(s) may include a memory configured to store data or other information associated with modules 910, 912, and 922, such as data storage/message buffer 916.

Storage device(s) 908 further include storage of a current cluster host network address 914 and factory address 918 assigned to end computing device 900 at the time of manufacture.

Event sensor(s) 904 may include any type of sensor(s), and the type of sensor(s) may depend at least in part upon the particular application in which the adaptive route network is to be deployed, and the type of event(s) which are to be detected. For example, sensor(s) 904 may include one or more sensors applicable in smart home, healthcare, artificial intelligence, transportation, government, automotive, commercial and/or industrial applications, among others. Event detection module 912 may include functionality that when executed by processor(s) 902, cause end computing device 900 to sense or detect one or more events, and/or perform any corresponding analysis or communication regarding such detected events, depending upon the requirements of the application in which the end devices are being implemented.

Transmission of certain types of messages by end computing device 900 are event triggered. For example, upon detection of an event by one of sensor(s) 904, processor(s) 902 may execute event detection module 912 to analyze the detected event data received from sensors 904, generate any corresponding data associated with the event (such as date and time stamps, etc.), and generate and transmit an event message including the event data and any corresponding data (all of which may be referred to herein simply as, “event data”). End computing devices 900 may include a message buffer 916 to buffer messages in the event they cannot be transmitted at the time of the event, or in those applications where events are transmitted on a batch basis, etc. In some examples, event detection module 912 may further include instructions that allow end computing device 900 to communicate with and/or analyze data received from other computing devices, such as electronic user identification badges, or with any other end devices computing devices (whether of the same type or a different type).

In a hand hygiene compliance network, for example, the system may include a plurality of compliance badges for monitoring of an individual user's hand hygiene practices. In some examples, each of a plurality of compliance badges is uniquely assigned to one of a plurality of users whose hand hygiene practices are to be monitored. The hand hygiene compliance system may further include dispenser module end computing devices, each of which detects dispense events at a hand hygiene product dispenser and communicates with compliance badges to associate each dispense event with a particular user. The system may further include zone module end computing devices, each of which generates a “zone” around an area to be monitored, and detects entry and/or exit events of compliance badges to/from the zone, such as a zone around a patient bed or other area to be monitored. To analyze compliance with hand hygiene procedures, the system includes one or more sets of compliance rules that define compliant and non-compliant hand hygiene practices. Upon sensing of a zone entry/exit event and/or dispense event, the sensing end device obtains badge identification information from the compliance badge associated with the zone entry/exit event and/or dispense event. The dispense event data and/or the zone entry/exit event data is transmitted from the end device(s) to the server, which analyzes the data in accordance with the compliance rules. In this way, individual compliance/non-compliance with hand hygiene procedures may be monitored and analyzed.

In accordance with the bi-directional communication protocols described herein, the event message will include the factory address 918 of end computing device 900. In some examples, the event message is transmitted to the current cluster host address 914 at the time of the occurrence of the event. Sending the event messages at the time of the event permits the server computing device to analyze the data and/or make decisions regarding the event in real time or near real time. This also helps to reduce memory requirements of each end computing device 900, as less memory is required to buffer or store large amounts of event data, thus increasing simplicity of design, reducing memory requirements, and reducing costs.

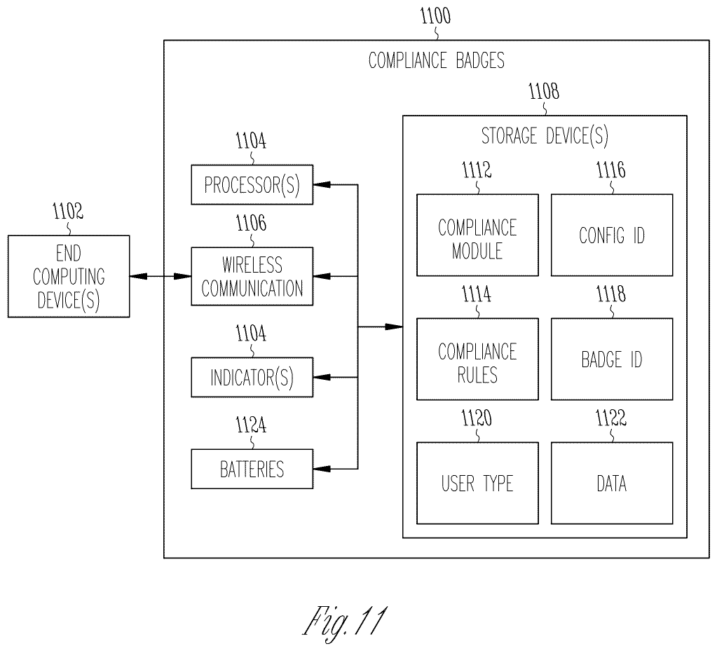

FIG. 10 is a block diagram of an example hand hygiene compliance monitoring system 1050 which uses an adaptive route network and bi-directional communication protocol in accordance with the present disclosure. A plurality of healthcare facilities, such as hospitals 1052A-1052N, each include a plurality of manual and/or touch free hand hygiene product dispensers for the dispensation of hand hygiene product(s). The hand hygiene product(s) may include antimicrobial soaps, hand sanitizers, general use soaps, surgical scrubs, lotions, body washes, etc. The hand hygiene products may take the form of any of liquids, gels, foams, lotions, pastes, powders, pellets, or any other form in which a hand hygiene product may be dispensed.

In this example, in order to monitor hand hygiene compliance of a plurality of healthcare workers associate with the healthcare facility, each healthcare worker is uniquely assigned to one of a plurality of compliance badges 1054A-1054N. For simplicity of illustration, these are shown with respect to hospital 1052A. It shall be understood, however, that compliance badges need not necessarily be used with implementation of an adaptive route network, but rather that compliance badges may be used in conjunction with an adaptive route network where monitoring of individuals is desired, such as the example shown in FIG. 10.

Each of the plurality of manual dispensers is associated with a different one of a plurality of manual dispenser end computing devices 1056A-1056N configured to detect a dispense event each time the respective manual dispenser is actuated. Similarly, each of the plurality of touch free dispensers is associated with a different one of a plurality of touch free dispenser end computing devices 1058A-1058N and is configured to detect a dispense event each time the respective touch free dispenser is actuated. In addition, each of a plurality of bed zone end computing device(s) 1060A-1060N is configured to generate a zone around an area to be monitored, such as a patient bed zone, and to detect entry events into the zone when it detects a compliance badge entering the patient bed zone, and to detect exit events out of the zone when it detects a compliance badge leaving the patient bed zone. Each of end computing devices 1056A-1056N, 1058A-1058N, and 1060A-1060N may be implemented as the end computing device 900 as shown in FIG. 9.

It shall be understood that other end computing devices associated with other devices, apparatus, and/or areas to be monitored may also be included, and that the disclosure is not limited in this respect. For example, end computing devices may also be associated with an area to be monitored, such as to detect presence of a compliance badge or healthcare worker in a patient room, treatment area, bathroom, or other area to be monitored). End computing devices may also be associated with any of sinks, toilets, or other device, apparatus, or area to be monitored for monitoring of hand hygiene compliance.

Each compliance badge 1054A-1054N is configured for short-range wireless communication with any of end computing device(s) 1056A-1056N, 1058A-1058N, and 1060A-1060N. Upon detection of a dispense event, for example, a manual dispenser end computing device 1056A-1056N may generate and transmit a short-range wireless interrogation signal, which induces any compliance badge 1054A-1054N within range of the transmission to transmit badge data, such as badge id, healthcare worker id, etc., upon receipt of the interrogation signal. The short-range wireless communication may include, for example short-range radio (RF) (e.g., Bluetooth, ZigBee, or ultra-wide band (UWB)) communication, infrared (IR) communication, or near field (NFC) communication techniques.

The end computing device(s) 1056A-1056N, 1058A-1058N, and 1060A-1060N are further configured to form part of an adaptive route wireless network 1070 and communicate using the bi-directional communication protocol in accordance with the present disclosure. Upon receipt of the badge data from a compliance badge, end computing device 1056A-1056N associates the badge data with the dispense event, and transmits the badge data along with the other dispense event data, in an adaptive route network message as described herein.

To that end, end computing device(s) 1056A-1056N, 1058A-1058N, and 1060A-1060N are configured for wireless transmission of dispense event data and/or entry/event data via the adaptive route network 1070. Each of end computing devices 1056A-1056N, 1058A-1058N, and 1060A-1060N is therefore configured to join a cluster with, and to transmit to and receive data from, one of cluster host computing devices 1062A-1062N. Each of cluster host computing devices 1062A-1062N is further in turn configured to join a route to a gateway 1064A-1064N with one or more of the other cluster host computing device 1062A-1062N as described herein (or none if there is only one cluster host along the route).

The dispense event data may include, among other things, a time and date stamp for the dispense event, a healthcare worker id or badge number received from a compliance badge associated with the dispense event, and a dispenser id. The dispense event data may also include status information corresponding to the dispense event, including a battery level for the dispenser or for the associated end computing device, a bottle presence indicator, a dispense event count, a number of dispenses remaining, a product empty indicator, or any other information relevant to the dispense event or to the status of the dispenser.

The zone entry/exit event data may include, among other things, a time and date stamp for the entry/exit event, a healthcare worker id or badge number received from a compliance badge associated with the entry/exit event, and a bed zone beacon id. The entry/exit event data may also include status information corresponding to the entry/exit event, including a battery level for the bed zone beacon or for the associated end computing device, an entry event count, an exit event count, or any other information relevant to the entry/exit event or to the status of the bed zone beacon or end computing device.

To monitor hand hygiene compliance, dispense event data from the plurality of dispenser end computing devices 1056A-1056N, 1058A-1058N, and/or entry/exit event data from the plurality of bed zone end computing device(s) 1060A-1060N, is wirelessly transmitted along a route through the adaptive route network in accordance with the bi-directional communication protocol of the present disclosure to one or more server computing device(s) 1082 for data analysis and reporting. Adaptive route network 1070 may communicate with server computing device(s) 1082 via one or more networks 1080. Network(s) 1080 may include, for example, one or more of a dial-up connection, a local area network (LAN), a wide area network (WAN), the internet, a wireless or Wi-Fi network, a cell phone network, satellite communication, or other means of electronic communication. The communication within network(s) 1080 may be wired or wireless. In addition, the local computing device(s) 1068, server computing device(s) 1082, and remote user computing device(s) 1084 may communicate via network(s) 1080. Remote/local computing device(s) 1084 may include, for example, one or more of a server computing device, a desktop computing device, a laptop computing device, a tablet computing device, a mobile computing device (such as a smart phone) a personal digital assistant, a pager, or any other type of computing device.

Server computing device 1082 includes an analysis application that, when executed by processors of server computing device 1082, analyzes the hand hygiene data (e.g., dispense event data and entry/exit event data) in accordance with one or more compliance rules so as to monitor hand hygiene compliance of healthcare workers within the healthcare facility. Server computing device 1082 further includes a reporting application that, when executed by processors of server computing device 1082, generates reports regarding hand hygiene compliance. For example, server computing devices 1082 may analyze the hand hygiene data to monitor hand hygiene compliance by individual healthcare worker, type of healthcare worker (e.g., nurses, doctors, environmental services (EVS), housekeeping personnel, maintenance personnel, etc.), department, type of department, individual hospital, type of hospital, across multiple hospitals, or by various other selected parameters. Server computing devices 1082 may generate and transmit a variety of reports automatically or on demand to one or more local computing device(s) 1068, one or more remote user computing device(s) 1084, with both qualitative and quantitative data regarding hand hygiene compliance at their hospital, to compare data over time to determine whether improvement has occurred, and/or to benchmark hand hygiene compliance at one hospitals, at multiple hospitals, or to view and compare hand hygiene compliance over time. Analysis and/or reporting applications may also be stored locally on hospital computing devices 1068 so that analysis and reporting of hand hygiene data may be done locally if desired.

In some example adaptive route networks in accordance with the present disclosure, the system may include a plurality of badges for monitoring user's behavior and/or interaction with other devices in the network. In a hand hygiene compliance network, for example, each of a plurality of compliance badges may be uniquely assigned to one of a plurality of users whose hand hygiene practices are to be monitored. The hand hygiene compliance system may further include dispenser module end computing devices, each of which detects dispense events at a hand hygiene product dispenser and communicates with the compliance badges to associate each dispense event with a particular user. The system may further include zone module end computing devices, each of which generates a “zone” around an area to be monitored, and detects entry and/or exit events of compliance badges to/from the zone, such as a zone around a patient bed or other area to be monitored.

To analyze compliance with hand hygiene procedures, the adaptive route network may include one or more sets of compliance rules that define compliant and non-compliant hand hygiene practices. Each set of compliance rules corresponds to a different type of user. In a hand hygiene network for use in a healthcare facility, for example, the user types may include physicians, nurses, physical therapists, environmental service staff, administrative personnel, etc. The compliance rules corresponding to each user type may include zone entry/exit event and dispense event timings designed with the anticipated workflow of the user type taken into account. Each set of compliance rules may include one or more configurable items that may be programmed or adjusted to accommodate the workflow of a corresponding user type. For example, the configurable items may include one or more audible indicator settings, one or more visible indicator settings, one or more timers or grace periods including times between patient zone entry/exit events, times between patient zone entry/exit events and dispense events, times after leaving a patient zone, and any other configurable item that may be used to evaluate compliance with hand hygiene procedures.

Upon sensing of a zone entry/exit event and/or dispense event, the sensing end device obtains badge identification information from the compliance badge associated with the zone entry/exit event and/or dispense event. The dispense event data and/or the zone entry/exit event data (including a time/date stamp associated with the event, the badge id, device id, etc.) is transmitted from the end device(s) to the server via the adaptive route network, which analyzes the data in accordance with the compliance rules. In this way, individual compliance/non-compliance with hand hygiene procedures may be monitored and analyzed. Each compliance badge may also be programmed to analyze dispense event and/or zone entry/exit data in accordance with the compliance rules to determine compliance/non-compliance with the hand hygiene procedures.

In some circumstances, it may be desirable to update, customize, or otherwise change the one or more sets of compliance rules or other badge settings in an adaptive route network. To that end, the adaptive route network includes configurable compliance badges having one or more sets of compliance rules that may be configured based on user type and/or the needs of the site.

The server computing device maintains a current set of compliance rules. Each time the set of compliance rules is updated or changed, the server increments a “configuration id” number corresponding to the current set of compliance rules. In order to distribute the updated set of compliance rules throughout the adaptive route network, the server transmits the current set of compliance rules and associated configuration id to the gateway computing device(s), which is local to the site.

To distribute the current set of compliance rules from a gateway computing device to all the hub/cluster host computing devices in an adaptive route network, the heartbeat messages transmitted by the route nodes (e.g., hub/cluster host computing device) in an adaptive route network are used to monitor the sets of compliance rules stored by each device. As mentioned above, in some examples, to verify that a route node is active (that is, that a route node is a hub with current network membership), each active route node (e.g., hub/cluster host computing device) transmits heartbeat messages at periodic (e.g., one (1) hour) intervals. Each heartbeat message includes the hub computing device's configuration id number. Each time the gateway receives a heartbeat message from a route node, the gateway compares the hub computing device's configuration id number with the current configuration id number. If the hub computing device's configuration id number is less than the current configuration id number, the gateway transmits a badge configuration message (BCM) to the hub computing device, and also transmits the current set of compliance rules and the current configuration id number to the device. In response to receipt of the BCM, the hub computing device updates the set of compliance rules and the configuration id number stored on the hub computing device. In this way, a current set of compliance rules may be distributed to all of the hubs in an adaptive route network using a relatively small amount of network traffic. As the heartbeat message is sent only once per hour, and the configuration id number takes up only a small number of bytes in the heartbeat message, the status of each device's set of compliance rules may be determined using a relatively small amount of network traffic, and the full set of compliance rules need only be sent if it is determined that a particular device's configuration id is less than the configuration id corresponding to the current set of compliance rules. In addition, the hubs may be updated relatively quickly as their configurations are checked with every heartbeat message.

Similarly, a dispense event message and/or heartbeat message transmitted by each end computing device (such as a dispenser) also includes the configuration id stored by the end computing device's stored configuration id. An end computing device generates a heartbeat message if no events have occurred within the heartbeat timeout period (e.g., 1 hour). If an event occurs before the heartbeat timeout has expired, the end computing device will reset the heartbeat timeout to 1 hour. Each time a hub/cluster host computing device receives a heartbeat message or dispense event message from an end computing device, the hub computing device compares its configuration id number with the configuration id number received from the end computing device. If the end computing device's configuration id number is less than the hub's configuration id number, the hub transmits a badge configuration message (BCM) to the end computing device, and also transmits the set of compliance rules and the configuration id number stored by the hub to the end computing device. In response to receipt of the BCM, the end computing device updates the set of compliance rules and the configuration id number stored on the end computing device. In this way, similar to updating the hubs in an adaptive route network, an updated set of compliance rules may be distributed to all of the end computing devices in an adaptive route network using a relatively small amount of network traffic. Because the configuration id number takes up only a small number of bytes in an event message or a heartbeat message, the status of each end computing device's set of compliance rules may be determined using a relatively small amount of network traffic, and the full set of compliance rules need only be sent if it is determined that a particular end device's configuration id is less than the configuration id stored by the hub/cluster host computing device. An end computing device will be checked at least as often as a heartbeat message is sent, and sometimes more frequently if a dispense event occurs.

To update the compliance badges in the network with the current set of compliance rules, each time an end computing device detects a dispense event or zone entry/exit event, the device establishes communication with the compliance badge associated with the event, and the compliance badge transmits a dispense event message, including its configuration id number and badge identification number to the end computing device. For example, if a dispenser detects a dispense event, the dispenser establishes communication with the compliance badge associated with the dispense event, and the compliance badge transmits dispense event message, including its badge id number and its configuration id number to the dispenser. The dispenser compares the configuration id number received from the compliance badge with the configuration id number stored by the dispenser. If the badge configuration id number is less than the configuration id number stored by the dispenser, the dispenser transmits a badge configuration message (BCM) back to the compliance badge associated with the dispense event. In response to receipt of the BCM, the badge updates the set of compliance rules and the configuration id number stored on the badge. In this way, the set of compliance rules stored by each compliance badge in the network is checked against the current set of compliance rules each time a dispense event associated with the badge is detected, and the set of compliance rules stored by the compliance badge is updated if necessary.

As with updating the hubs and the end computing devices, an updated set of compliance rules may be distributed to all of the compliance badges in an adaptive route network using a relatively small amount of network traffic. Because the configuration id number takes up only a small number of bytes in an event message, the status of each compliance badge's set of compliance rules may be determined using a relatively small amount of network traffic, and the full set of compliance rules need only be sent if it is determined that a particular compliance badge's configuration id is less than the configuration id stored by the end computing device.

The time required for updating a compliance badge is determined by how often the compliance badge is used to complete a dispense event.

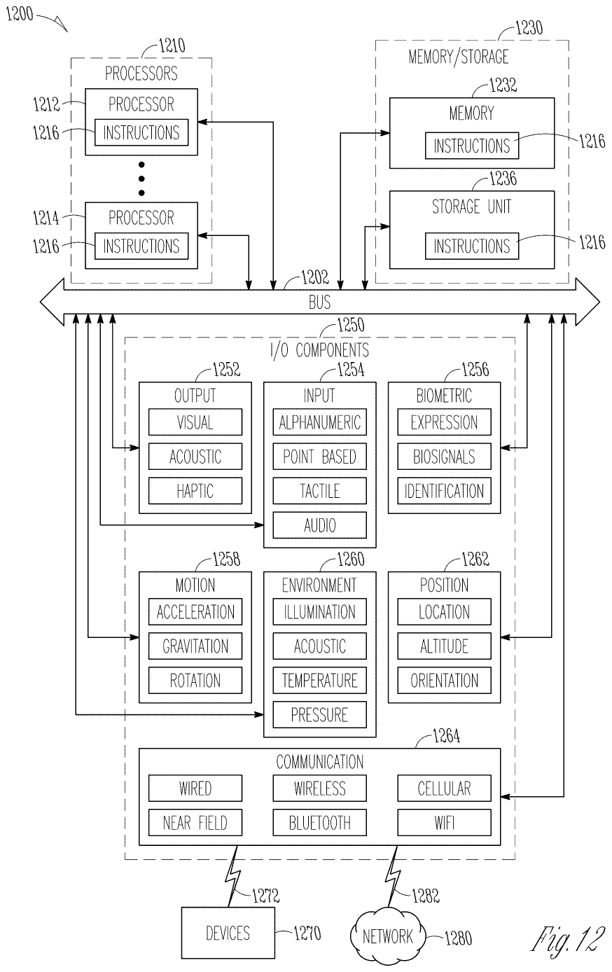

FIG. 11 is a block diagram of an example compliance badge 1100 in accordance with the adaptive route network and protocol of the present disclosure. FIG. 11 illustrates one example of a compliance badge 1100, and many other examples of compliance badge 1100 may be used in other instances. Compliance badge 1100 may include a subset of the components included in FIG. 11 or may include additional components not shown in FIG. 11.

Compliance badge 1100 includes one or more processor(s) 1104, a wireless communication unit 1106, one or more indicator(s) 1110, one or more batteries 1124, and one or more storage device(s) 1108. Storage device(s) 1108 includes a compliance module 1112, compliance rules 1114, a configuration id 1116, a badge id 1118, a user type 1120, and a data storage/message buffer 1122.

One or more wireless communication units 1106 permit short-range wireless communication with end computing devices, such as end computing device(s) 1102 in an adaptive route network. The end computing device(s) 1102 may include, for example, any of dispenser end devices and/or bed beacon end devices in a hand hygiene compliance network, or any other type of end computing device. Upon detection of a dispense event, for example, a dispenser end computing device 1102 may generate and transmit a short-range wireless interrogation signal, which induces any compliance badge 1100 within range of the transmission to transmit badge data, such as badge id, healthcare worker id, configuration id number, etc., in response to receipt of the interrogation signal. The short-range wireless communication may include, for example short-range radio (RF) (e.g., Bluetooth, ZigBee, or ultra-wide band (UWB)) communication, infrared (IR) communication, or near field (NFC) communication techniques.

One or more processors 1104 may implement functionality and/or execute instructions associated with compliance badge 1100. Examples of processors 1104 include application processors, microcontrollers, and any other hardware configured to function as a processor, a processing unit, controller, or a processing device. Processors 1104 may execute compliance module 1112 to communicate with one or more end computing device(s), detect dispense and/or exit/entry events, and/or perform any corresponding analysis or communication regarding such detected events. For example, processors 1104 may retrieve and execute instructions stored by storage components 1108 that cause processors 1104 to perform or execute the operations stored in compliance module 1112. The instructions, when executed by processors 1104, may cause compliance badge 1100 to generate and/or store information within storage components 1108, such as data storage/message buffer 1122.

In some examples, compliance rules 1114 includes one or more sets of compliance rules, each corresponding to a different defined user type. In a healthcare facility, for example, the user types may include a physician user type, a nurse user type, a physical therapist user type, an environmental services user type, a dietary stuff user type, and any other defined user type. Each compliance is uniquely associated with a different user, and the user type 1120 corresponding to that user is stored. To analyze hand hygiene behaviors, compliance badge 1100 uses the set of compliance rules 1114 corresponding to the user type 1120.

Transmission of certain types of messages by compliance badge 1100 may be event triggered. For example, upon detection of an event by one of end computing devices 1102, processor(s) 1104 may execute compliance module 1112 to generate and transmit an event message including badge id 1118, user type 1120, and configuration id 1116 to the end computing device 1102 associated with the event. Compliance module 1112 may also include instructions that, when executed by processor(s) 1104, permit compliance badge 1100 to analyze data corresponding to the detected event based on the compliance rules 1114 and the user type 1120 to determine compliance and/or non-compliance with hand hygiene practices.

### Modules, Components, And Logic

The PZM and badges of the present subject matter operate on hardware for control, calculations, and wireless communications. They may be embodied in hardware, software, firmware, and combinations thereof. Certain embodiments may be include logic or a number of components, modules, or mechanisms. Modules may constitute either software modules (e.g., code embodied on a machine-readable medium) or hardware modules. A “hardware module” is a tangible unit capable of performing certain operations and may be configured or arranged in a certain physical manner. In various example embodiments, one or more computer systems (e.g., a standalone computer system, a client computer system, or a server computer system) or one or more hardware modules of a computer system (e.g., a processor or a group of processors) may be configured by software (e.g., an application or application portion) as a hardware module that operates to perform certain operations as described herein.

In some embodiments, a hardware module may be implemented mechanically, electronically, or any suitable combination thereof. For example, a hardware module may include dedicated circuitry or logic that is permanently configured to perform certain operations. For example, a hardware module may be a special-purpose processor, such as a Field-Programmable Gate Array (FPGA) or an Application Specific Integrated Circuit (ASIC). A hardware module may also include programmable logic or circuitry that is temporarily configured by software to perform certain operations. For example, a hardware module may include software executed by a general-purpose processor or other programmable processor. Once configured by such software, hardware modules become specific machines (or specific components of a machine) uniquely tailored to perform the configured functions and are no longer general-purpose processors. It will be appreciated that the decision to implement a hardware module mechanically, in dedicated and permanently configured circuitry, or in temporarily configured circuitry (e.g., configured by software) may be driven by cost and time considerations.

Accordingly, the phrase “hardware module” should be understood to encompass a tangible entity, be that an entity that is physically constructed, permanently configured (e.g., hardwired), or temporarily configured (e.g., programmed) to operate in a certain manner or to perform certain operations described herein. As used herein, “hardware-implemented module” refers to a hardware module. Considering embodiments in which hardware modules are temporarily configured (e.g., programmed), each of the hardware modules need not be configured or instantiated at any one instance in time. For example, where a hardware module comprises a general-purpose processor configured by software to become a special-purpose processor, the general-purpose processor may be configured as respectively different special-purpose processors (e.g., comprising different hardware modules) at different times. Software accordingly configures a particular processor or processors, for example, to constitute a particular hardware module at one instance of time and to constitute a different hardware module at a different instance of time.

Hardware modules can provide information to, and receive information from, other hardware modules. Accordingly, the described hardware modules may be regarded as being communicatively coupled. Where multiple hardware modules exist contemporaneously, communications may be achieved through signal transmission (e.g., over appropriate circuits and buses) between or among two or more of the hardware modules. In embodiments in which multiple hardware modules are configured or instantiated at different times, communications between such hardware modules may be achieved, for example, through the storage and retrieval of information in memory structures to which the multiple hardware modules have access. For example, one hardware module may perform an operation and store the output of that operation in a memory device to which it is communicatively coupled. A further hardware module may then, at a later time, access the memory device to retrieve and process the stored output. Hardware modules may also initiate communications with input or output devices, and can operate on a resource (e.g., a collection of information).

The various operations of example methods described herein may be performed, at least partially, by one or more processors that are temporarily configured (e.g., by software) or permanently configured to perform the relevant operations. Whether temporarily or permanently configured, such processors may constitute processor-implemented modules that operate to perform one or more operations or functions described herein. As used herein, “processor-implemented module” refers to a hardware module implemented using one or more processors.

Similarly, the methods described herein may be at least partially processor-implemented, with a particular processor or processors being an example of hardware. For example, at least some of the operations of a method may be performed by one or more processors or processor-implemented modules. Moreover, the one or more processors may also operate to support performance of the relevant operations in a “cloud computing” environment or as a “software as a service” (SaaS). For example, at least some of the operations may be performed by a group of computers (as examples of machines including processors), with these operations being accessible via a network (e.g., the Internet) and via one or more appropriate interfaces (e.g., an Application Program Interface (API)).

The performance of certain of the operations may be distributed among the processors, not only residing within a single machine, but deployed across a number of machines. In some example embodiments, the processors or processor-implemented modules may be located in a single geographic location (e.g., within a home environment, an office environment, or a server farm). In other example embodiments, the processors or processor-implemented modules may be distributed across a number of geographic locations.

### Machine And Software Architecture

The modules, methods, applications and so forth described in conjunction with the figures of this application are implemented in some embodiments in the context of a machine and an associated software architecture. The sections below describe a representative architecture that is suitable for use with the disclosed embodiments.

Software architectures are used in conjunction with hardware architectures to create devices and machines tailored to particular purposes. For example, a particular hardware architecture coupled with a particular software architecture will create a mobile device, such as a mobile phone, tablet device, or so forth. A slightly different hardware and software architecture may yield a smart device for use in the “internet of things.” While yet another combination produces a server computer for use within a cloud computing architecture. Not all combinations of such software and hardware architectures are presented here as those of skill in the art can readily understand how to implement the invention in different contexts from the disclosure contained herein.

### Example Machine Architecture And Machine-Readable Medium

FIG. 12 is a block diagram illustrating components of a machine 2300, according to some example embodiments, able to read instructions from a machine-readable medium (e.g., a machine-readable storage medium) and perform any one or more of the methodologies discussed herein. Specifically, FIG. 12 shows a diagrammatic representation of the machine 1200 in the example form of a computer system, within which instructions 1216 (e.g., software, a program, an application, an applet, an app, or other executable code) for causing the machine 1200 to perform any one or more of the methodologies discussed herein may be executed. Additionally, or alternatively, the instructions may implement one or more of the devices and/or components of the present subject matter. The instructions transform the general, non-programmed machine into a particular machine programmed to carry out the described and illustrated functions in the manner described. In alternative embodiments, the machine 1200 operates as a standalone device or may be coupled (e.g., networked) to other machines. In a networked deployment, the machine 1200 may operate in the capacity of a server machine or a client machine in a server-client network environment, or as a peer machine in a peer-to-peer (or distributed) network environment. The machine 1200 may comprise, but not be limited to, a server computer, a client computer, a personal computer (PC), a tablet computer, a laptop computer, a netbook, a personal digital assistant (PDA), or any machine capable of executing the instructions 1216, sequentially or otherwise, that specify actions to be taken by machine 1200. Further, while only a single machine 1200 is illustrated, the term “machine” shall also be taken to include a collection of machines 1200 that individually or jointly execute the instructions 1216 to perform any one or more of the methodologies discussed herein.

The machine 1200 may include processors 1210, memory/storage 1230, and I/O components 1250, which may be configured to communicate with each other such as via a bus 1202. In an example embodiment, the processors 1210 (e.g., a Central Processing Unit (CPU), a Reduced Instruction Set Computing (RISC) processor, a Complex Instruction Set Computing (CISC) processor, a Graphics Processing Unit (GPU), a Digital Signal Processor (DSP), an Application Specific Integrated Circuit (ASIC), a Radio-Frequency Integrated Circuit (RFIC), another processor, or any suitable combination thereof) may include, for example, processor 1212 and processor 1214 that may execute instructions 1216. The term “processor” is intended to include multi-core processor that may comprise two or more independent processors (sometimes referred to as “cores”) that may execute instructions contemporaneously. Although FIG. 12 shows multiple processors, the machine 1200 may include a single processor with a single core, a single processor with multiple cores (e.g., a multi-core process), multiple processors with a single core, multiple processors with multiples cores, or any combination thereof.

The memory/storage 1230 may include a memory 1232, such as a main memory, or other memory storage, and a storage unit 1236, both accessible to the processors 1210 such as via the bus 1202. The storage unit 1236 and memory 1232 store the instructions 1216 embodying any one or more of the methodologies or functions described herein. The instructions 1216 may also reside, completely or partially, within the memory 1232, within the storage unit 1236, within at least one of the processors 1210 (e.g., within the processor's cache memory), or any suitable combination thereof, during execution thereof by the machine 1200. Accordingly, the memory 1232, the storage unit 1236, and the memory of processors 1210 are examples of machine-readable media.

As used herein, “machine-readable medium” means a device able to store instructions and data temporarily or permanently and may include, but is not limited to, random-access memory (RAM), read-only memory (ROM), buffer memory, flash memory, optical media, magnetic media, cache memory, other types of storage (e.g., Erasable Programmable Read-Only Memory (EEPROM)) and/or any suitable combination thereof. The term “machine-readable medium” should be taken to include a single medium or multiple media (e.g., a centralized or distributed database, or associated caches and servers) able to store instructions 1216. The term “machine-readable medium” shall also be taken to include any medium, or combination of multiple media, that is capable of storing instructions (e.g., instructions 1216) for execution by a machine (e.g., machine 1200), such that the instructions, when executed by one or more processors of the machine 1200 (e.g., processors 1210), cause the machine 1200 to perform any one or more of the methodologies described herein. Accordingly, a “machine-readable medium” refers to a single storage apparatus or device, as well as “cloud-based” storage systems or storage networks that include multiple storage apparatus or devices. The term “machine-readable medium” excludes signals per se.

The I/O components 1250 may include a wide variety of components to receive input, provide output, produce output, transmit information, exchange information, capture measurements, and so on. The specific I/O components 1250 that are included in a particular machine will depend on the type of machine. For example, portable machines such as mobile phones will likely include a touch input device or other such input mechanisms, while a headless server machine will likely not include such a touch input device. It will be appreciated that the I/O components 1250 may include many other components that are not shown in FIG. 12. The I/O components 1250 are grouped according to functionality merely for simplifying the following discussion and the grouping is in no way limiting. In various example embodiments, the I/O components 1250 may include output components 1252 and input components 1254. The output components 1252 may include visual components (e.g., a display such as a plasma display panel (PDP), a light emitting diode (LED) display, a liquid crystal display (LCD), a projector, or a cathode ray tube (CRT)), acoustic components (e.g., speakers), haptic components (e.g., a vibratory motor, resistance mechanisms), other signal generators, and so forth. The input components 1254 may include alphanumeric input components (e.g., a keyboard, a touch screen configured to receive alphanumeric input, a photo-optical keyboard, or other alphanumeric input components), point based input components (e.g., a mouse, a touchpad, a trackball, a joystick, a motion sensor, or other pointing instrument), tactile input components (e.g., a physical button, a touch screen that provides location and/or force of touches or touch gestures, or other tactile input components), audio input components (e.g., a microphone), and the like.

In further example embodiments, the I/O components 1250 may include biometric components 1256, motion components 1258, environmental components 1260, or position components 1262 among a wide array of other components. For example, the biometric components 1256 may include components to detect expressions (e.g., hand expressions, facial expressions, vocal expressions, body gestures, or eye tracking), measure biosignals (e.g., blood pressure, heart rate, body temperature, perspiration, or brain waves), identify a person (e.g., voice identification, retinal identification, facial identification, fingerprint identification, or electroencephalogram based identification), and the like. The motion components 1258 may include acceleration sensor components (e.g., accelerometer), gravitation sensor components, rotation sensor components (e.g., gyroscope), and so forth. The environmental components 1260 may include, for example, illumination sensor components (e.g., photometer), temperature sensor components (e.g., one or more thermometer that detect ambient temperature), humidity sensor components, pressure sensor components (e.g., barometer), acoustic sensor components (e.g., one or more microphones that detect background noise), proximity sensor components (e.g., infrared sensors that detect nearby objects), gas sensors (e.g., gas detection sensors to detection concentrations of hazardous gases for safety or to measure pollutants in the atmosphere), or other components that may provide indications, measurements, or signals corresponding to a surrounding physical environment. The position components 1262 may include location sensor components (e.g., a Global Position System (GPS) receiver component), altitude sensor components (e.g., altimeters or barometers that detect air pressure from which altitude may be derived), orientation sensor components (e.g., magnetometers), and the like.

Communication may be implemented using a wide variety of technologies. The I/O components 1250 may include communication components 1264 operable to couple the machine 1200 to a network 1280 or devices 1270 via coupling 1282 and coupling 1272 respectively. For example, the communication components 1264 may include a network interface component or other suitable device to interface with the network 1280. In further examples, communication components 1264 may include wired communication components, wireless communication components, cellular communication components, Near Field Communication (NFC) components, Bluetooth® components (e.g., Bluetooth® Low Energy), Wi-Fi® components, and other communication components to provide communication via other modalities. The devices 1270 may be another machine or any of a wide variety of peripheral devices (e.g., a peripheral device coupled via a Universal Serial Bus (USB)).

Moreover, the communication components 1264 may detect identifiers or include components operable to detect identifiers. For example, the communication components 1264 may include Radio Frequency Identification (RFID) tag reader components, NFC smart tag detection components, optical reader components (e.g., an optical sensor to detect one-dimensional bar codes such as Universal Product Code (UPC) bar code, multi-dimensional bar codes such as Quick Response (QR) code, Aztec code, Data Matrix, Dataglyph, MaxiCode, PDF413, Ultra Code, UCC RSS-2D bar code, and other optical codes), or acoustic detection components (e.g., microphones to identify tagged audio signals). In addition, a variety of information may be derived via the communication components 1264, such as, location via Internet Protocol (IP) geo-location, location via Wi-Fi® signal triangulation, location via detecting a NFC beacon signal that may indicate a particular location, and so forth.

### Transmission Media

In various example embodiments, one or more portions of the network 1280 may be an ad hoc network, an intranet, an extranet, a virtual private network (VPN), a local area network (LAN), a wireless LAN (WLAN), a wide area network (WAN), a wireless WAN (WWAN), a metropolitan area network (MAN), the Internet, a portion of the Internet, a portion of the Public Switched Telephone Network (PSTN), a plain old telephone service (POTS) network, a cellular telephone network, a wireless network, a Wi-Fi® network, another type of network, or a combination of two or more such networks. For example, the network 1280 or a portion of the network 1280 may include a wireless or cellular network and the coupling 1282 may be a Code Division Multiple Access (CDMA) connection, a Global System for Mobile communications (GSM) connection, or other type of cellular or wireless coupling. In this example, the coupling 1282 may implement any of a variety of types of data transfer technology, such as Single Carrier Radio Transmission Technology (1×RTT), Evolution-Data Optimized (EVDO) technology, General Packet Radio Service (GPRS) technology, Enhanced Data rates for GSM Evolution (EDGE) technology, third Generation Partnership Project (3GPP) including 3G, fourth generation wireless (4G) networks, fifth generation wireless (5G) networks, Universal Mobile Telecommunications System (UMTS), High Speed Packet Access (HSPA), Worldwide Interoperability for Microwave Access (WiMAX), Long Term Evolution (LTE) standard, others defined by various standard setting organizations, other long range protocols, or other data transfer technology.

The instructions 1216 may be transmitted or received over the network 1280 using a transmission medium via a network interface device (e.g., a network interface component included in the communication components 1264) and utilizing any one of a number of well-known transfer protocols (e.g., hypertext transfer protocol (HTTP)). Similarly, the instructions 1216 may be transmitted or received using a transmission medium via the coupling 1272 (e.g., a peer-to-peer coupling) to devices 1270. The term “transmission medium” shall be taken to include any intangible medium that is capable of storing, encoding, or carrying instructions 1216 for execution by the machine 1200, and includes digital or analog communications signals or other intangible medium to facilitate communication of such software.

### Examples

The following listing of examples are demonstrative of the present subject matter and are only some of the examples possible given the teachings of the present subject matter and are not intended to limit the scope of the present subject matter.

Example 1 is a system including a patient zone monitor (PZM) including an ultra-wideband transceiver (UWB transceiver) including a low noise amplifier, an antenna in communication with the low noise amplifier of the UWB transceiver, and at least one processor in communication with the UWB transceiver, the processor programmed to control the low noise amplifier to set a range of communications of the PZM.

Example 2 is the system of Example 1, further comprising machine readable instructions executable by the processor to adjust the range of communications of the PZM.

Example 3 is the system of Example 1, wherein the PZM further includes a ground plane to reflect at least some of the communications from the antenna.

Example 4 is the system of Example 1, wherein the PZM is configured to be mounted on a wall at a head of a patient bed.

Example 5 is the system of Example 1, wherein the UWB transceiver is configured to operate in a frequency range of approximately 3.1 to 10.6 GHz.

Example 6 is the system of Example 1, wherein the range of communications creates a substantially elliptical shaped patient zone.

Example 7 is the system of Example 6, wherein the patient zone includes a patient zone boundary that is configurable and can be set to a default range or adjusted during installation.

Example 8 is the system of Example 1, further comprising a second antenna in communication with the UWB transceiver.

Example 9 is the system of Example 1, wherein the processor is further programmed to adjust a gain setting of the low noise amplifier to modify the range of communications of the PZM within a healthcare environment.

Example 10 is the system of Example 1, wherein the processor is programmed to save different low noise amplifier settings corresponding to different patient zone ranges.

Example 11 is the system of Example 1, wherein the processor is programmed to control the low noise amplifier to create different sensitivity zones for monitoring hand hygiene compliance.

Example 12 is a method for determining patient zone ranges in a healthcare environment, comprising: transmitting, by a patient zone monitoring device (PZM), an ultra-wideband (UWB) signal to a range set for the healthcare environment; receiving, by the PZM, a response signal from a portable radio device; and determining the portable radio device is within the range of the PZM.

Example 13 is the method of Example 12, comprising creating a shaped patient zone using a directional antenna.

Example 14 is the method of Example 13, further comprising configuring a patient zone boundary to a default range or adjusting the patient zone boundary during installation.

Example 15 is the method of Example 14, wherein the patient zone boundary includes at least one hand hygiene dispenser.

Example 16 is the method of Example 14, further comprising transmitting patient zone boundary data from the PZM to the portable radio device.

Example 17 is the method of Example 12, wherein the UWB signal is transmitted in a frequency range of approximately 3.1 to 10.6 GHz.

Example 18 is the method of Example 14, further comprising differentiating signals received from in front of the PZM from signals received from behind the PZM using an antenna of the PZM.

Example 19 is the method of Example 14, further comprising detecting a hand hygiene event when the portable radio device is determined to be within a hand hygiene dispenser zone.

Example 20 is the method of Example 14, wherein the portable radio device is a compliance badge worn by a healthcare worker.

Example 21 is a system including a patient zone monitoring device (PZM) with an ultra-wideband transceiver (UWB transceiver), a first antenna and a second antenna in communication with the UWB transceiver, and at least one processor in communication with the UWB transceiver. The processor is programmed to determine information relating to an angle-of-arrival (AoA) and a time-of-flight (ToF) using communications by the UWB transceiver with a portable radio device.

Example 22 is the system of Example 21, further including machine readable instructions, wherein when the processor executes the instructions it can determine whether the portable radio device is within one patient zone range of a plurality of patient zone ranges using the determined information relating to AoA and ToF.

Example 23 is the system of Example 22, further including instructions executable by the processor to identify at least one patient zone range of the plurality of patient zone ranges that it determines the portable radio device is within.

Example 24 is the system of Example 21, wherein the PZM is configured to be mounted on a wall at a head of a patient bed.

Example 25 is the system of Example 21, wherein the UWB transceiver is configured to operate in a frequency range of approximately 3.1 to 10.6 GHz.

Example 26 is the system of Example 21, wherein the processor is further programmed to determine a distance between the PZM and the portable radio device using the ToF information.

Example 27 is the system of Example 22, wherein the plurality of patient zone ranges creates a patterned patient zone.

Example 28 is the system of Example 27, wherein the patient zone includes a patient zone boundary that is configurable and can be set to a default range or adjusted during installation.

Example 29 is the system of Example 22, wherein the processor is further programmed to create a shaped patient zone by associating different boundary ranges with different arrival angles.

Example 30 is the system of Example 21, further including a third antenna in communication with the UWB transceiver, wherein the processor is programmed to use the three antennas to differentiate signals from in front of the PZM from signals from behind the PZM.

Example 31 is a method for determining patient zones in a healthcare environment, including: transmitting, by a patient zone monitoring device (PZM), an ultra-wideband (UWB) signal; receiving, by the PZM, a response signal from a portable radio device; determining, by a processor of the PZM, angle-of-arrival (AoA) information based on the received response signal; determining, by the processor, time-of-flight (ToF) information based on a time difference between the transmitted UWB signal and the received response signal; calculating, by the processor, a distance between the PZM and the portable radio device based on the ToF information; defining, by the processor, a plurality of patient zones based on the calculated distance and the AoA information, wherein each patient zone is associated with a different range of AoA values; and determining, by the processor, which of the plurality of patient zones the portable radio device is located within based on the calculated distance and the AoA information.

Example 32 is the method of Example 31, further including associating each of the plurality of patient zones with a different range of AoA values.

Example 33 is the method of Example 31, wherein defining the plurality of patient zones includes creating a shaped patient zone by associating different boundary ranges with different arrival angles.

Example 34 is the method of Example 31, further including configuring a patient zone boundary to a default range or adjusting the patient zone boundary during installation.

Example 35 is the method of Example 31, wherein the plurality of patient zones includes at least one hand hygiene dispenser zone.

Example 36 is the method of Example 31, further including transmitting patient zone boundary data from the PZM to the portable radio device.

Example 37 is the method of Example 31, wherein the UWB signal is transmitted in a frequency range of approximately 3.1 to 10.6 GHz.

Example 38 is the method of Example 31, further including differentiating signals received from in front of the PZM from signals received from behind the PZM using a third antenna in communication with the UWB transceiver.

Example 39 is the method of Example 31, further including detecting a hand hygiene event when the portable radio device is determined to be within a hand hygiene dispenser zone.

Example 40 is the method of Example 31, wherein the portable radio device is a compliance badge worn by a healthcare worker.

Example 41 is at least one machine-readable medium including instructions that, when executed by processing circuitry, cause the processing circuitry to perform operations to implement of any of Examples 1-40.

Example 42 is an apparatus comprising means to implement of any of Examples 1-41.

Example 43 is a system to implement of any of Examples 1-42.

Example 44 is a method to implement of any of Examples 1-43.

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. The scope of the invention should be determined with reference to the appended claims, along with the full scope of equivalents to which such claims are entitled.

What is claimed is:

1. A system, comprising:
a patient zone monitor (PZM) including an ultra-wideband transceiver (UWB transceiver) including a low noise amplifier, an antenna in communication with the a low noise amplifier of the UWB transceiver, and at least one processor in communication with the UWB transceiver, the processor programmed to control the low noise amplifier to set a range of communications of the PZM.

2. The system of claim 1, further comprising:
machine readable instructions executable by the processor to adjust the range of communications of the PZM.

3. The system of claim 1, wherein the PZM further includes a ground plane to reflect at least some of the communications from the antenna.

4. The system of claim 1, wherein the PZM is configured to be mounted on a wall at a head of a patient bed.

5. The system of claim 1, wherein the UWB transceiver is configured to operate in a frequency range of approximately 3.1 to 10.6 GHz.

6. The system of claim 1, wherein the range of communications creates a substantially elliptical shaped patient zone.

7. The system of claim 6, wherein the patient zone includes a patient zone boundary that is configurable and can be set to a default range or adjusted during installation.

8. The system of claim 1, further comprising a second antenna in communication with the UWB transceiver.

9. The system of claim 1, wherein the processor is further programmed to adjust a gain setting of the low noise amplifier to modify the range of communications of the PZM within a healthcare environment.

10. The system of claim 1, wherein the processor is programmed to save different low noise amplifier settings corresponding to different patient zone ranges.

11. The system of claim 1, wherein the processor is programmed to control the low noise amplifier to create different sensitivity zones for monitoring hand hygiene compliance.

12. A method for determining patient zone ranges in a healthcare environment, comprising:
transmitting, by a patient zone monitoring device (PZM), an ultra-wideband (UWB) signal to a range set for the healthcare environment;
receiving, by the PZM, a response signal from a portable radio device; and
determining the portable radio device is within the range of the PZM.

13. The method of claim 12, comprising:
creating a shaped patient zone using a directional antenna.

14. The method of claim 13, further comprising:
configuring a patient zone boundary to a default range or the patient zone boundary during installation.

15. The method of claim 14, wherein the patient zone boundary includes at least one hand hygiene dispenser.

16. The method of claim 14, further comprising:
transmitting patient zone boundary data from the PZM to the portable radio device.

17. The method of claim 12, wherein the UWB signal is transmitted in a frequency range of approximately 3.1 to 10.6 GHz.

18. The method of claim 14, further comprising:
differentiating signals received from in front of the PZM from signals received from behind the PZM using an antenna of the PZM.

19. The method of claim 14, further comprising:
detecting a hand hygiene event when the portable radio device is determined to be within a hand hygiene dispenser zone.

20. The method of claim 14, wherein the portable radio device is a compliance badge worn by a healthcare worker.
